(19) United States
(12) Patent Application Publication
(10) Pub. No.: US 2004/0014814 A1
(43) Pub. Date: Jan. 22, 2004

(54) Compositions and methods for delaying preventing rejuvenating or reversing senescence in cells

(76) Inventors: Geoff W. Grigg, Linley Point (AU); Peter Molloy, Campbell (AU)

Correspondence Address: FOLEY AND LARDNER, SUITE 500, 3000 K STREET NW, WASHINGTON, DC, 20007, US

(21) Appl. No.: 10/381,057

(22) PCT Filed: Sep. 25, 2001
(86) PCT No.: PCT/AU01/01199

(30) Foreign Application Priority Data
Sep. 26, 2000 (AU) PR 0382

(51) Int. Cl. A61K 31/198

(52) U.S. Cl. 514/563

(57) ABSTRACT

A method of altering the senescence of cells comprising applying to said cells an effective amount of a composition wherein said composition includes N-acetyl-carnosine as an active ingredient.

### INTRODUCTION TO THE INVENTION

This invention relates to the field of cell senescence and in particular relates to the determination of unexpected properties of N-acetyl-carnosine in the delay, prevention, rejuvenation or reversal of cell senescence particularly in skin cells, without contributing to cell toxicity. Selected aspects of the invention include compositions and methods for accomplishing or improving on the above affects.

### BACKGROUND OF THE INVENTION

Aging of cells, tissues, and whole animals, including man, is associated with a diminution in function of tissues and organs and an overall decline in the ability to prevent damage to cells and tissues by toxins and to repair such damage once it occurs. A number of factors both intrinsic and extrinsic, influence the rate of aging. One of the most important of the latter is the level of insult by oxidative radicals and one of the most important intrinsic factors is the level of protective agents present in the aging tissue. This generalisation on aging is true also for the skin. Skin, however, has to contend with another major toxic agent, solar radiation. Solar radiation greatly increases the rate of aging of skin primarily due to the ability of its shorter wavelength components to induce the production of oxidative radicals in the skin and their subsequent reaction with cell organelles and macromolecules. As a direct consequence, skin collagen is both degraded and cross-linked, lipids are peroxidated, DNA is modified, and toxic Advanced Glycation End products (AGEs) are produced.

L-Carnosine is a dipeptide consisting of β-alanine linked at its carboxyl end to the amino group of L-histidine (β-alanyl-L-histidine). It is synthesized by the enzyme carnosine synthetase, and broken down by the enzyme carnosinase. Carnosine was first identified in beef extract in 1900. Since then, carnosine and related compounds have been shown to be present in millimolar concentrations in several mammalian tissues, including skeletal muscle, cardiac muscle, olfactory lobe of the brain and the crystalline lens of the eye. It has been established that the three main imidazole dipeptides found in mammalian tissues are carnosine, anserine (β-alanyl-i-methyl-L-histidine) and balenine (β-alanyl-3-methyl-L-histidine). More recently acetylated forms of these histidyl compounds have been reported in muscle and brain tissues. There is still little understanding of the biological roles of these compounds, however, in recent times some putative roles as well as applications have been described. These include regulation of glycogenolysis, neurotransmission in the brain, muscle activation, wound healing, treatment of gastric ulcers, antioxidant activity and the inhibition of nonenzymatic glcosylation of proteins.

Recent studies have shown that senescent MRC-5 cells transferred to a medium containing carnosine (20 mM) were rejuvenated (see PCT/AU91/00541). This was observed by cells acquiring a younger phenotype and showing an increase in cell growth and proliferation. It has been shown that there is a correlation between carnosine concentration in skeletal muscle and species longevity. One may deduce that one of the ways carnosine and its analo gues function as antioxidants in slowing down the aging process is through effects on one or more of the processes described above. One of the problems of carnosine as a therapeutic agent, however, is its extreme lability in body fluids such as blood because of the presence in those fluids of peptidase such as carnosinase, which cleave carnosine into its constituent amino acids. In addition it is known that carnosine is taken up into cells only slowly. Interestingly, some of the carnosine analogues are found in higher concentrations in mammalian tissues than carnosine itself. For instance in rat muscle the concentration of anserine is between 4 to 6 times higher than carnosine and in guinea pig cardiac muscle the concentration of N-acetyl carnosine is 8 time that of carnosine. Similar concentration differences are found in human muscle.

In addition it has been found that acylderivatives of carnosine are both effective in blocking erythema and its attendant oedema induced by skin exposure to solar radiation.

Conventional wisdom indicated that a lipophilic derivative should be taken up into the cell more rapidly than the parent compound so one of the objectives of this project was to improve the up-take characteristics of carnosine into cells and into the skin whilst retaining its intrinsic anti-ageing properties intact by modifying the basic structure to make it more lipophilic. In order to achieve this aim a number of carnosine analogues were constructed having an increased lipid solubility, which it was hoped would lead to increased cell uptake kinetics and which also would be able to delay, prevent, rejuvenate or reverse senescence in cells without exhibiting undesirable toxicity.

Carnosine has a low intrinsic toxicity to normal skin cells except those which have undergone a malignant transformation (Holliday and Mcfarland (1996) Brit. J. Canc., 73,966) so that it was surprising that when a representative series of both N-acyl and ester derivatives were synthesised and tested by the present inventors, a substantial proportion were either toxic or interfered with normal growth in some way or other. One derivative, however, N-acetyl carnosine, promoted cell growth well. In cell uptake trials despite being more lipophilic than carnosine, N-acetyl-carnosine performed rather worse than carnosine, contrary to expectations. Surprisingly, however, despite its inferior cell uptake performance, it exhibited superior anti-ageing properties in a number of ways. Thus it slowed down senescence of late passage fibroblasts in culture and rejuvenated senescent cells.

Longevity growth curves showed that N-acetyl carnosine allowed more population doublings (PDs) than controls and significantly more PDs than carnosine itself. Moreover, immunohistochemical staining using monoclonal antibodies against the cyclin-dependant kinase inhibitor p16^{INK4/CDKN2} showed that this protein was not expressed in young or non-senescent cells but was expressed in senescent cells. Treatment with N-acetyl carnosine led to reduced expression of this biomarker of senescence as well as restoration of the juvenile morphology and growth habit.

Accordingly, it is believed that N-acetyl-carnosine has properties useful in slowing down, or reversing aging in normal and diseased cells or rejuvenating senescent cells which are superior to those of carnosine. Such properties would also provide numerous benefits for the treatment of age and degenerative disease states, and provide benefits for the cosmetic industry.

Unlike carnosine, N-acetyl carnosine was found by the present inventors to be almost completely resistant to attack by human blood peptidases. Another interesting and unexpected property of the N-acetyl compound was that once in the cell it enjoyed a considerably longer residence time than did carnosine before each was degraded, the former to carnosine, and the latter to histidine and beta alinine. The greater biological activity, and other unexpected properties of this compound leading to the current invention may be understood from these unexpected observations.

### STATEMENT OF INVENTION

Accordingly, in one aspect the invention provides a method of altering the senescence of cells comprising applying to said cells an effective amount of a composition where said composition includes N-acetyl-carnosine as an active ingredient.

The alteration of said senescence may include any one of a combination of: delaying the onset of senescence, preventing senescence, reversing senescence or rejuvenating senescent cells.

In another aspect the invention provides a method of treating age or degenerative related disease in a subject suffering from same comprising administering to said subject an effective amount of a composition which includes N-acetyl-carnosine as an active ingredient.

In another aspect the invention provides a method of rejuvenating skin, assisting the skin in its ability to renew itself and/or reducing aging of skin of a subject comprising administering to said subject an effective amount of a composition which includes N-acetyl-carnosine as an active ingredient.

In another aspect the invention provides a method for slowing down aging of skin and the development of those features which characterise skin aging such as changes in skin texture, changes in pigmentation or discolouration, diminution of immunoreactiveness, increased sensitivity to the toxic and genotoxic effects of environmental toxins including solar radiation.

In another aspect the invention provides a method for treating skin inflammatory conditions arising from stimuli such as exposure to allergens, solar radiation, or skin infections.

In another aspect the invention provides a method of increasing cell mass.

In another aspect the invention provides a pharmaceutical composition characterised by the inclusion of N-acetyl carnosine as a actual ingredient. Preferably N-acetyl-carnosine is used at a concentration between 1 mM to 20 mM.

In another aspect the invention provides for the administration to cells, the skin or other epidermis, N-acetyl carnosine in a suitable transdermal formulation of which there are many, ranging from simple oil-in-water emulsions, or water-oil emulsions, to aqueous solutions containing a specific transdermal agent such as salicylate or polyethylene glycol. The preferred formulation is an oil-in-water emulsion.

### DETAILED DESCRIPTION OF THE INVENTION

In order that the nature of the invention may be more fully understood some particularly preferred forms thereof will be described with reference to the following examples and figures in which:-FIG. 1 shows the cumulative growth of MRC-5 cells in DMEM supplemented with carnosine (20 mM) or various carnosine analogues (5 mM) commencing with Passage 55 cells. Cultures were split in a 1:4 ratio when flasks were approximately 70-80% confluent.

FIG. 2 shows the cumulative growth of MRC-5 cells in DMEM supplemented with carnosine (20 mM) or acetyl-carnosine (5 mM). Cultures were split in a 1:4 ratio when flasks were approximately 70-80% confluent.

FIG. 3 shows the uptake of radio-labeled (3H) carnosine and (3H) acetyl-carnosine by MRC-5 fibroblast cells (A) or corneal epithelial (B).

FIG. 4 shows results indicative of rejuvenated cells.

FIG. 5 shows expression of the cyclin-dependent kinase inhibitor p16 in MRC-5 cell nuclei. Immunohistochemical staining showed no expression in young or non-senescent cells (A) but once cells reach senescence (B) a proportion of cells express p16. Senescent cells treated with acetyl-carnosine acquire a younger phenotype and no longer express p16 (C). Cells were stained from passage 30 until senescence was reached (approximately passage 65) in order to determine the effectiveness of p16 as a reliable biomarker.

Cell Culture

MRC -5 cells (fetal lung fibroblasts) were grown in Dulbecco's modification of Eagles' minimum essential medium (DMEM Gibco BRL Cat. No. 12800-017) containing 0.45% glucose, or MEM (Gibco BRL Cat. No. 612800-017) containing 0.1% glucose. Both media contained 10% fetal calf scrum (TRACE Biosciences Cat. No. 15-010-0500V) and Penicillin/Streptomycin 5000 IU, 5000 μg/ml (TRACE Biosciences Cat. No. 21-140-0100V). Cells were grown in 25 cm³ flasks or in dishes (35 mm or 60 mm, Nunc) at 37° C. with 5% CO₂. Cells were harvested with trypsin (TRACE Biosciences Cat. No. 21-163-0100V) and counted with a model ZF6 Coulter Counter. Cells were split in ratios of 1:2 or 1:4. Population doublings (PDs) were calculated from the ratio of the initial number of cells plated to the yield of cells after a period of time. This provides an accurate measure of cell growth, with the split ratios 1:2 and 1:4 corresponding to one and two passages respectively, which are broadly equivalent to population doublings. The media was changed every 5 days or when cells became confluent.

Uptake studies

Radio-labeled (3H) carnosine and acetyl-carnosine were assayed for their rate of uptake using fresh bovine eyes and MRC-5 fibroblast cultures. For bovine eyes small cylinders (radius 3.5mm and height 8mm) placed on top of the eye formed a fix seal with the corneal epithelial cells. 200 microlitres of the radio-labeled compounds in 5mM unlabeled compound in phosphate buffered saline were instilled onto the eye and at different time points 600 μl of aqueous humour was aspirated using a 25 gauge needle for counting in the scintillation counter.

For MRC-5 cells, (3H)—carnosine and acetyl carnosine were added to the medium of near confluent 60mm culture dishes. At different time points cells were washed three times with phosphate buffered saline, cells harvested and cell-associated radioactivity determined using Packard Starscint scintillation fluid.

Immunohistochemical Staining Procedure

Tissue culture cells were fixed by slowly infusing 4% paraformaldehyde in 0.1M sodium phosphate buffer pH 7.4 into the culture medium. This solution was then replaced with fixative alone and stored at −20° C. for 30 minutes. Cultures were then defrosted for 5 minutes at 37° C. and then washed with 3 changes of the 0.1M sodium phosphate buffer.

Tissue cultures were then treated with 3% H₂O₂ in phosphate buffer for 5 minutes to quench endogenous peroxidase activity and then sequentially incubated with the following reagents: (i) primary antibody for 45 minutes at room temperature; (ii) biotinylated link antibody (Universal Large Volume DAKO LSAB+Kit, Peroxidase Cat. No. K0690, DAKO Australia) for 30 minutes at room temperature; (iii) peroxidase-labeled streptavidin (Universal Large Volume DAKO LSAB+ Kit, Peroxidase Cat. No. K0690, DAKO Australia) for 30 minutes at room temperature; (iv) Substrate-chromogen solution (DAKO Large Volume DAB+ Cat. No. K3468, DAKO Australia) for 5 minutes at room temperature. All immunoreagents were diluted with 0.1M phosphate buffer pH 7.4 and cell cultures were washed three times in 0.1M phosphate buffer between steps (5 minute washes).

Three primary antibodies were used in this study: (i) murine monoclonal antibodies against cyclin-dependent kinase inhibitor p27^{kipl} (clone SX53G8, IgG, Loto38, DAKO, diluted 1:200); (ii) murine monoclonal antibodies against cyclin-dependent kinase inhibitor p21^{WAP/CIPI} (clone SX118, IgG, Loto88, DAKO, diluted 1:200); and (iii) murine monoclonal antibodies against cyclin-dependent kinase inhibitor p16^{INK4/CDKN2} (clone DCS-50, IgG, Lot 077H4821, Sigma, diluted 1:200). The cell culture dishes were counterstained with Hematoxylin. There was no staining of cultures when the primary antibody was omitted from the reaction sequence. An estimate of positively stained cells to specific antigens was determined.

### EXAMPLE 1

MRC-5 cells were used to observe the toxicity of a series of derivatives of carnosine on cell growth. Cell cultures were grown in DMEM media with 10% fetal calf serum and supplemented with the following carnosine analogues: carnosine ethyl ester, carnosine iso-propyl ester, carnosine n-butyl ester, carnosine n-hexyl ester, N-propyl carnosine, N-hexanoyl carnosine, N-trimethyl acetyl carnosine, N-acetyl carnosine methyl ester, N-acetyl carnosine ethyl ester and N-acetyl carnosine, at concentrations ranging between 1 mM to 20 mM. Cells were also grown in DMEM supplemented with carnosine at 20 mM. Toxicity studies showed that ester analogues formed at the carboxyl terminus of the carnosine molecule (carnosine ethyl ester, carnosine iso-propyl ester, carnosine n-butyl ester, carnosine n-hexyl ester, N-acetyl carnosine methyl ester, N-acetyl carnosine ethyl ester) were toxic to cell growth at concentrations above 2 mM (see Table 1). Even at lower concentrations cell morphology was that of cells that have reached senescence i.e. broad flat cells with long process reaching out to other cells. Cell numbers were less than in the control flasks. No toxicity was observed with the other carnosine analogues. Hence, it was decided to test the effect of carnosine and these non toxic carnosine analogues on the lifespan of MRC-5 cells in culture commencing with cells at passage 55 (Table 2). As shown in FIG. 1 Acetyl-carnosine achieved a lifespan 5 PDs greater than the control and 6.7 PDs greater than carnosine over a 43 day period. In fact acetyl-carnosine markedly increased the lifespan of MRC-5 cells in culture compared with the other carnosine analogues. In the early phase of the experiment growth rates were markedly greater than with the other compounds (FIG. 1). After 43 days cell growth in all the other flasks had either reached a plateau or started to decrease, however, the flask containing acetyl-carnosine showed cell growth continuing.

TABLE 1
Effect of carnosine derivatives on cell growth.
(0) Toxic to cell growth, (10) Confluent cell growth.

| | 0.5 mM | 1 mM | 2 mM | 5 mM | 10 mM | 20 mM | |
|---|---|---|---|---|---|---|---|
| Control | — | — | — | — | — | — | 6 |
| N-acetyl carnosine | 7 | 8 | 9 | 10 | 9 | 8 | — |
| Carnosine | — | — | — | 5 | 6 | 7 | — |
| Carnosine ethyl ester | — | — | 5 | 5 | 4 | 3 | — |
| Carnosine iso-propyl | — | — | 2 | 0 | 0 | 0 | — |
| ester | | | | | | | |
| Carnosine n-butyl | — | — | 5 | 5 | 2 | 0 | — |
| ester | | | | | | | |
| carnosine n-hexyl | — | — | 3 | 1 | 0 | 0 | — |
| ester | | | | | | | |
| N-propyl carnosine | 6 | 6 | 5 | 4 | 3 | 2 | — |
| N-hexanoyl | 6 | 6 | 5 | 4 | 3 | 3 | — |
| carnosine | | | | | | | |
| N-trimethyl acetyl | 6 | 6 | 5 | 4 | 3 | 3 | — |
| carnosine | | | | | | | |
| N-acetyl carnosine | 6 | 6 | 5 | 5 | 5 | 0 | — |
| methyl ester | | | | | | | |
| N-acetyl carnosine | 6 | 5 | 4 | 4 | 2 | 0 | — |
| ethyl ester | | | | | | | |
| Capric | — | 2 | 0 | 0 | — | — | — |
| Palmatoyl | — | 3 | — | — | — | — | — |

TABLE 2
The effect of carnosine and some of its analogues of the lifespans of
MRC-5 cells in culture, expressed as Population Doublings (PD). The
experiment was commenced using cells at passage 55.

| Compound | PD |
|---|---|
| Control | 10.2 |
| Carnosine (20 mM) | 8.5 |
| Acetyl-carnosine (5 mM) | 15.2 |
| TMAA (5 mM) | 9.4 |
| Hexanoyl (5 mM) | 10.8 |
| Propanoyl (5 mM) | 11.0 |

Data shown in FIG. 2 demonstrated the acetyl-carnosine, even at lower concentrations, markedly increased the lifespan of MRC-5 cells in culture compared with carnosine; this experiment commenced with younger cells at passage32. In fact all populations of MRC-5 cells grown in DMEM supplemented with acetyl-carnosine preserved a younger phenotype for much longer than cells grown in DMEM supplemented with carnosine or its analogues. Cell cultures grown in carnosine showed signs of senescence far earlier than cells grown in acetyl-carnosine which maintained a non-senescent morphology for far longer and increased the lifespan of cells in culture.

The results in this example demonstrate the variability of lifespans of populations of MRC-5 cells when exposed to carnosine or carnosine analogues. Under control conditions the range was 57.3-64.5 PDs (n=5). With the addition of carnosine (20mM) to the medium the range was 61.8-63.7 PDs (n=5) with an average increase of 1.4 PDs which represents a 3 fold increase in cell number. However, when acetyl-carnosine (5 mM) was added to the medium, the range was 62.3-70.3 PDs (n=5) with an average increase of 5 PDs compared with the controls. This represents a 32 fold increase in cell number. These results indicate that both carnosine and acetyl-carnosine extend the chronological lifespan of MRC-5 cells in culture, however, this extension is markedly increased with media supplemented with acetyl-carnosine.

### EXAMPLE 2

The rate of uptake of carnosine and acetyl-carnosine by MRC-5 cells was studied using radio-labeled tritiated (3H) carnosine and acetyl-carnosine compounds. The uptake was measured in MRC-5 cells at passage 28 over a 48 hour period (FIG. 3A). Data demonstrates that after 48 hours the uptake of carnosine was 2 times greater than the uptake of acetyl-carnosine i.e. 0.32% vs 0.16% respectively. However, interestingly, after 48 hours the number of cells growing in media supplemented with acetyl-carnosine (3.4×10⁶) was 42% greater than the number of cells growing in media supplemented with carnosine (2.4×10⁶). Thus uptake of labeled acetyl carnosine was 35% of that of carnosine on a per cell basis.

### EXAMPLE 3

The uptake experiments were repeated using fresh bovine eyes. As shown in FIG. 3B, after 4 hours the uptake of carnosine was 2 times greater than the uptake of acetyl-carnosine i.e. 1.3% vs o.6% respectively. The results show that the uptake rate of carnosine and acetyl-carnosine from the corneal epithelial cells into the aqueous humour is far greater than the uptake rate in MRC-5 fibroblasts. However, in both types the relative uptake of carnosine and acetyl-carnosine is comparable.

### EXAMPLE 4

It was shown above that acetyl-carnosine markedly delayed the onset of senescence by increasing the lifespan of MRC-5 cells in culture. This was confirmed by rejuvenation experiments (FIG. 4). The morphology of cells grown in media supplemented with acetyl-carnosine was distinct from the controls. In control flasks cells were broad and flat with long processes extending to other cells (FIG. 4A). In fact some cells began to extend bipolar processes. Cells became very granular and debris accumulated in the medium. This morphology was typical of senescent cells. However, cells grown in media supplemented with acetyl-carnosine were long and spindle and gave an appearance of streaming (FIG. 4B). Cell growth showed confluency and cells acquired a phenotype typical of younger cells.

To confirm this rejuvenation of senescent cells by acetyl-carnosine we observed the expression of 3 known biomarkers of aging cells. These biomarkers were p21, p27 and p16, are all cyclin-dependent kinase inhibitors and are expressed at different stages of the cell cycle. Table 3 shows the expression of these biomarkers in both senescent and non-senescent MRC-5 fibroblast cells. The proteins p21 and p27 show a similar staining profile in the nucleus in both senescent and non-senescent cells. In both cases non -senescent cultures show about 30% of nuclei unstained while essentially all nuclei stain in senescent cultures. However, p16 provides a cleaner biomarker of senescence as immunohistochemical staining using monoclonal antibodies against p16^{INK4/CDKN2} showed that this protein was not expressed in young or non-senescent cells (FIG. 5A) but was expressed in a significant proportion of senescent cells (FIG. 5B). In the rejuvenation experiment once cells started to exhibit some degree of nuclei staining indicating senescence (FIG. 5B), old media was replaced with media supplemented with acetyl-carnosine. After a few days the cells began to adopt a younger phenotype and there was no expression of p16 in the nucleus (FIG. 5C).

TABLE 3
The expression patterns of the cyclin-dependant kinase inhibitors p21,
p27 and p16 in the nucleus of MRC-5 human fetal lung fibroblasts.

| | | Stained | Unstained |
|---|---|---|---|
| P 21 | (non-senescent) | 70% | 30% |
| | (senescent) | >99% | <1% |
| P 27 | (non-senescent) | 70% | 30% |
| | (senescent) | >99% | <1% |
| P 16 | (non-senescent) | 0% | 100% |
| | (senescent) | 40% | 60% |

TABLE 4
A comparison of the various properties of carnosine derivatives.
Explanation of symbols: 0 = zero effect, 10 = maximum effect.

| Comparison | | | | | | UV immuno | | | |
|---|---|---|---|---|---|---|---|---|---|
| of properties | | | | | UV anti- | suppression: | | Cell and | |
| of carnosine | | anti- | anti- | | inflamma- | post | pre- | eye | |
| and | Cell | oxidant | glycation | Rejuvenation | tory assay | irradiation | irradiation | uptake | Lifespan |
| derivatives | Growth | (1) | (2) | Assay | (3) | assay (3) | assay (3) | assay | extension |
| Control | − | | | | | | | | |
| N-acetyl | +++++ | +++ | 0 | +++++++ | ++++ | ++++++ | ++++++ | ++ | ++++ |
| carnosine | | | | | | | | | |
| Carnosine | +++ | ++++ | +++++ | +++ | ++++ | 0 | +++ | ++++ | (++) |
| Carnosine | +++ | | | | | | | | |
| ethyl ester | | | | | | | | | |
| Carnosine | TOXIC | | | | | | | | − |
| iso-propyl | | | | | | | | | |
| ester | | | | | | | | | |
| Carnosine n- | TOXIC | | | | | | | | − |
| butyl ester | | | | | | | | | |
| carnosine n- | TOXIC | | | | | | | | − |
| hexyl ester | | | | | | | | | |
| N-propyl | ++ | | | | | | | | 0 |
| carnosine | | | | | | | | | |
| N-hexanoyl | ++ | | | | | | | | 0 |
| carnosine | | | | | | | | | |
| N-trimethyl | ++ | | | | | | | | 0 |
| acetyl | | | | | | | | | |
| carnosine | | | | | | | | | |
| N-acetyl | + | | | | | | | | − |
| carnosine | | | | | | | | | |
| methyl ester | | | | | | | | | |
| N-acetyl | + | | | | | | | | − |
| carnosine | | | | | | | | | |
| ethyl ester | | | | | | | | | |
| Capric | TOXIC | | | | | | | | − |
| Palmatoyl | TOXIC | | | | | | | | − |

(1) Babizhayev, M. A. Biochim. Biophys. Acta 1989; 1004:363-371; Dahl, T. A., et al, Photochem. Photobiol., 1988; 47:357-362.
(2) Hipkiss, A. R. et al FEBS Lett. 1995; 371:81-85.
(3) Grigg, G. W., Australian Provisional Patent Application No. PQ 5150 19 Jan. 2000, Treatment of UV-Induced Immuno-suppression

From the foregoing, the invention can be seen to provide an effective method and composition for altering cell senescence.

Human diploid fibroblasts originating from fetal lung tissue pass through three phases of growth in vitro. Phase I or the primary culture terminates with the formation of the first confluent layer of cells. Phase II is characterised by a prolific cell growth requiring many subcultivations. Finally cells enter Phase III or senescence and are typically large and irregular in shape with long process reaching out to other cells. At this stage cells have lost their young phenotypic morphology. The current invention demonstrates that acetyl-carnosine, when used as an active ingredient in the treatment of cells, not only markedly extends the lifespan of cells, particularly MRC-5 fibroblasts in vitro, but also rejuvenates cells that have just reached senescence or are in the early stages of Phase III. The examples and disclosures of the invention show that as control or non-treated cells become senescent the acetyl-carnosine treated cells have gained a phenotype typical of younger cells. A comparison of longevity curves between acetyl-carnosine treated cells and non-treated cells illustrates firstly that the most marked differences in cell growth occurred in the mid to late stages of Phase II and secondly that the onset of Phase III was retarded in acetyl-carnosine treated cells. Carnosine supplemented media was shown to both increase the life span of MRC-5 fibroblasts in vitro and rejuvenate senescent cells.

What is unexpected however, is the observation that acetyl carnosine, despite exhibiting substantially lower kinetics of uptake into an intact epithelium of the bovine eye and into human fibroblasts in culture, was consistently more effective in its antiageing activity than carnosine, a fact which could not have been predicted, particularly given the poor performance in this regard by other, closely related acyl carnosine derivatives. Moreover, unlike carnosine, acetyl carnosine exhibited a lower performance in two characteristics thought to be important in the ageing process, via., non-enzymic glycation and antioxidant capacity, yet exhibited superior overall effectiveness in the cell senescence assays. In tests acetyl-carnosine proved ineffective as an antiglycation agent and possessed only 60% of the antioxidant effectiveness of the parent compound.

This invention also demonstrates a 3-fold increase in cell mass when cells were exposed to carnosine supplemented media. This increase is less than the 12-fold increase that reported by McFarland & Holliday. Interestingly, acetyl-carnosine applied to cells produced a 32-fold increase in cell mass. Longevity curves also showed that when cell growth in control flasks or even in flasks containing carnosine treated cells was reaching a plateau or in some cases decreasing, the cell growth in flasks containing media supplemented with acetyl-carnosine was still increasing linearly. These results indicate both the lifespan variability of MRC-5 fibroblasts in vitro and the marked efficacy of acetyl-carnosine in extending the chronological lifespan of MRC-5 fibroblasts compared with controls, carnosine and other carnosine analogues, some of which proved extremely toxic to cell growth.

The immunohistochemical staining patterns demonstrated that the two cyclin-dependent kinase inhibitors P21 and p27 have the same expression in both senescent and non-senescent cells. On average approximately 70% of young or non-senescent cells showed staining indicating that the cells are in different stages of the cell cycle and so the two proteins are not concurrently expressed in all cells. This differs from senescent cells in which the proteins are not concurrently expressed in all cells. This differs from senescent cells in which the proteins are expressed in all cells. Because of the high degree of staining in both cell types it was felt that distinguishing senescent from non-senescent cells was still largely morphologically based, making p21 and p27 not reliable biomarkers of senescence. However, fortuitously the third cyclin-dependent kinase inhibitor p16 proved to be a much more reliable biomarker of senescence as it was not expressed in young cells but was expressed in varying degrees in senescent cells.

Uptake studies using both MRC-5 fibroblasts and fresh bovine eyes demonstrated that the uptake of tritiated carnosine was two times greater than the uptake of tritiated acetyl-carnosine. Despite demonstrating that the acetyl-carnosine possesses greater biological activity than carnosine, this result was rather surprising as it had been anticipated that a greater efficacy would be associated with a higher rate of uptake. Experiments were conducted to determine the fate of acetyl-carnosine and carnosine after cellular uptake. Data revealed that label from carnosine was more rapidly incorporated into higher molecular weight proteins, suggesting that carnosine was much more susceptible to cleavage by endogenous enzymes. A slower metabolism and incorporation into other proteins may be one reason for acetyl-carnosine having higher effective intracellular concentrations compared with carnosine despite its lower overall uptake.

Of the many derivatives and analogues of carnosine examined in this and previous studies by ourselves and by Dr. R.Holliday and his associates (McFarland and Holliday, 1999, Exper. Gerentology 34:35-45), N-acetyl carnosine is the only one whose anti-aging and general cell and tissue protective properties exceed those of carnosine. Moreover we have observed that it is quite stable in human blood serum, unlike carnosine, which is rapidly destroyed.

Aging of cells in tissue culture is recognized as closely reflecting the aging of similar types of cells in normal biological tissues ( Holliday, R., Understanding Aging. Cambridge University Press, 1995 ).Thus the phenomenon we have described using human skin fibroblasts in the examples here has direct application to aging of these same cells in the intact skin.

A further benefit of the invention is the economy of the active ingredient N-acetyl-carnosine, which is active at lower concentrations than alternative actives and can therefore be formulated at a lower dosage cost with superior performance than currently know techniques.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

In conclusion this invention has provided for the first time acetyl-carnosine based compositions and methods which retard the onset of senescence and rejuvenate cells that have just become senescent.

Such compositions and methods find numerous applications including medical, therapeutic, preventative and cosmetic applications.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of altering the senescence of cells comprising applying to said cells an effective amount of a composition wherein said composition includes N-acetyl-carnosine as an active ingredient.

2. A method of altering the senescence of cells including any one or a combination of delaying the onset thereof, preventing or reversing senescence of cells or rejuvenating senescent cells comprising applying to said cells an effective amount of a composition including N-acetyl-carnosine as an active ingredient.

3. A method of treating aging or degenerative related diseases in a subject suffering from same comprising applying to said cells an effective amount of a composition including N-acetyl-carnosine as an active ingredient.

4. A method of treating ageing or degenerative related diseases in a subject suffering from same comprising applying to said cells an effective amount of a composition including N-acetyl-carnosine as an active ingredient.

5. A method of slowing down aging of skin and the development of those features which characterize skin aging including skin texture, changes in pigmentation or discolouration, diminution of immunoreactiveness, increased sensitivity to toxic and genotoxic effects of environmental toxins including solar radiation comprising applying to said cells an effective amount of a composition including N-acetyl-carnosine as an active ingredient.

6. A method of treating skin inflammatory conditions arising from stimuli such as exposure to allergies, solar radiation or skin infection comprising applying a to said cells an effective amount of a composition including N-acetyl-carnosine as an active ingredient.

7. A method of increasing cell mass comprising applying to said cells an effective amount of a composition including N-acetyl-carnosine as an active ingredient.

8. A method according to any one of claims 1 to 7 wherein said effective amount of N-acetyl-carnosine is provided at a concentration between 1 mM and 20 mM in said composition.

9. A method according to any one of claims 1 to 8 wherein said cells are human fibroblast cells.

10. A method according to claim 9 wherein said fibroblast cells are MRC-5 human fetal lung fibroblasts.

11. A method according to any one of claims 1 to 10 where the application is by administration to cells, the skin on other epidermis in a suitable transdermal formulation.

12. A method according to claim 11 wherein said formulation is selected from oil-in-water emulsions, water-oil emulsions, or aqueous solutions.

13. A method according to claim 12 wherein said aqueous solution contains a specific transdermal agent such as salicylate or polyethylene glycol.

14. A pharmaceutical composition comprising N-acetyl-carnosine as an active ingredient in combination with a pharmaceutically acceptable carrier.

15. A pharmaceutical composition according to claim 14 having N-acetyl-carnosine at a concentration between 1 mM and 20 mM.

16. A pharmaceutical composition according to claim 14 or 15 when used in the methods of any one of claims 1 to 10.

17. Use of N-acetyl-carnosine for the preparation of a composition or medicament for treatment according to one or a combination of the methods claimed in claims 1 to 13.

18. A method according to any one of claims 1 to 13 substantially as hereinbefore described with particular reference to the examples.

19. A pharmaceutical composition according to any one of claims 14 to 16 substantially as hereinbefore described with reference to the examples.

20. Use according to claim 17 substantially as hereinbefore described with reference to the examples.
